(19) United States
(12) Patent Application Publication
(10) Pub. No.: US 2024/0335679 A1
(43) Pub. Date: Oct. 10, 2024

(54) TOROIDAL GANTRY FOR A PARTICLE THERAPY SYSTEM

(71) Applicant: Mevion Medical Systems, Inc., Littleton, MA (US)

(72) Inventors: Mark Jones, Reading, MA (US); Gerrit Townsend Zwart, Durham, NH (US); James Cooley, Andover, MA (US)

(73) Assignee: Mevion Medical Systems, Inc., Littleton, MA (US)

(21) Appl. No.: 18/580,478

(22) PCT Filed: Jul. 15, 2022
(86) PCT No.: PCT/US2022/073772

§ 371 Date: Jan. 18, 2024

Related U.S. Application Data
(60) Provisional application No. 63/223,764, filed on Jul. 20, 2021.

(51) Int. Cl. A61N 5/10; G21K 5/04; H05H 13/02; H05H 13/04

(52) U.S. Cl. CPC A61N 5/1078; A61N 5/1048; A61N 5/1081; G21K 5/04; H05H 13/02; H05H 13/04; A61N 2005/1087; H05H 2277/11

(57) ABSTRACT

An example particle therapy system includes a particle accelerator configured to output a particle beam at a predefined maximum energy and a toroidal gantry comprising magnets in an interior thereof. The magnets include a first magnet proximate to an output of the particle accelerator and second magnets proximate to a treatment position. The first magnet is configured to direct the particle beam to a second magnet. The second magnet is configured to bend the particle at the predefined maximum energy towards the treatment position.

### TECHNICAL FIELD

This specification describes examples of particle therapy systems and gantries for use therein.

### BACKGROUND

Particle therapy systems use a particle accelerator to generate a particle beam for treating afflictions, such as tumors. Particle therapy systems may use a gantry to direct the particle beam toward a patient. In some examples, a gantry includes a device that supports a radiation delivery apparatus during treatment.

### SUMMARY

An example particle therapy system includes a particle accelerator configured to output a particle beam at a predefined maximum energy and a toroidal gantry comprising magnets in an interior thereof. The magnets include a first magnet proximate to an output of the particle accelerator and second magnets proximate to a treatment position. The first magnet is configured to direct the particle beam to a second magnet. The second magnet is configured to bend the particle at the predefined maximum energy towards the treatment position. The particle therapy system may include one or more of the following features either alone or in combination.

The particle accelerator and the toroidal gantry may be within a same treatment space. The particle accelerator may be a fixed-energy particle accelerator. The particle therapy system may include an energy degrader that is movable between each of the second magnets and the treatment position. The energy degrader may be configured to change an energy of the particle beam before the particle beam reaches the treatment position. The second magnets may be spaced apart and each may be located in a different circumferential sector of the toroidal gantry. The toroidal gantry may include between six and twenty second magnets. The second magnets may be stationary on the toroidal gantry. The second magnets many be configured to bend the particle beam by at least 90°. The particle therapy system may include a treatment couch that is movable within a hole of the toroidal gantry. The treatment couch may be for holding a patient at the treatment position. A distance between the second magnet and the treatment position may be two meters (2 m) or less. A distance between the second magnet and the treatment position may be one meter (1 m) or less. A distance between the second magnet and the treatment position may be 0.5 m or less.

The particle accelerator may be or include a synchrocyclotron. The particle accelerator may be or include a synchrocyclotron configured to operate at two energies, with one of the two energies being greater than another of the two energies. The particle accelerator may be or include a synchrotron.

The particle therapy system may include one or more imaging devices mounted to the toroidal gantry. The one or more imaging devices may be configured for movement around the toroidal gantry. The particle therapy system may include a nozzle that is configured for movement around the toroidal gantry. The nozzle may be for conditioning and outputting the particle beam to the treatment position. The particle therapy system may include a control system programmed to control movement of the one or more imaging devices and to control movement of the nozzle. The control system may be programmed to prevent collision between the nozzle and the one or more imaging devices. The nozzle may be configured to rotate around a first inner track in the toroidal gantry and the one or more imaging device may be configured to rotate around a second inner track in the toroidal gantry. The first inner track and the second inner track may be at different locations of the toroidal gantry.

The second magnets may be spaced apart and each may be located in a different circumferential sector of the toroidal gantry. Each of the sectors may include a nozzle for outputting the particle beam to the treatment position. The particle accelerator may include main superconducting coils to generate a magnetic field for accelerating particles to produce the particle beam. The particle accelerator may include active return coils to conduct current in an opposite direction as in the main superconducting coils. The particle beam may be delivered to the patient at FLASH doses. The particle beam may be delivered to the patient at a dose that exceeds twenty (20) Gray-per-second for a duration of less than five (5) seconds.

Another example particle therapy system includes a multi-sectored gantry, with each sector being configured to deliver radiation to a patient from a different position on the multi-sectored gantry, and a particle accelerator connected to the multi-sectored gantry to output the radiation towards the multi-sectored gantry. The multi-sectored gantry and the particle accelerator may be in a same treatment room and not separated by shielding external to the multi-sectored gantry or the particle accelerator. The particle therapy system may include one or more of the following features either alone or in combination.

The multi-sectored gantry and the particle accelerator may be in a same treatment space. Each sector may include a magnet configured to direct the radiation towards the patient. Each magnet may be substantially D-shaped. Each magnet may be configured to bend the particle beam by at least 90°. The multi-sectored gantry may be toroidal in shape. The multi-sectored gantry may include a second magnet in each sector and a first magnet between the second magnet and the particle accelerator. The first magnet may be for directing the particle beam to a second magnet in a target sector. The first magnet may be configured to direct the particle beam to different sectors. The particle accelerator may be or include a synchrocyclotron. The synchrocyclotron may be configured to output the particle beam at one of two different energies. The particle accelerator may be or include a synchrotron.

An example gantry for use in a particle therapy system includes a toroidal structure that is connectable to a particle accelerator. The toroidal structure includes first magnets arranged in sectors around a circumference of the toroidal structure. The first magnets are for bending a particle beam originating at the particle accelerator by at least 90° towards a treatment position. An enclosure connects the toroidal structure to the particle accelerator. The enclosure includes second magnets. The second magnets are for receiving the particle beam and for directing the particle beam towards the first magnets. A rotatable structure within the enclosure is configured for mounting at least one of radiation delivery components or imaging components. The gantry may be within a same treatment space as the particle accelerator.

The gantry may include an energy degrader that is movable between each of the first magnets and the treatment position. The energy degrader may be configured to change an energy of the particle beam before the particle beam reaches the treatment position. The energy degrader may be mounted to the rotatable structure.

The first magnets may be spaced apart and are each located in a different circumferential sector of the toroidal structure. The toroidal structure may include between six and twenty first magnets. The first magnets may be stationary on the toroidal structure. The second magnets may be configured to bend the particle beam by at least 90°. A distance between each of the first magnets and the treatment position may be two meters (2 m) or less. A distance between the second magnet and the treatment position may be one meter (1 m) or less.

The gantry may include one or more imaging devices mounted to the rotatable structure. The one or more imaging devices may be configured for movement around the toroidal structure. The gantry may include a nozzle that is configured for movement around the toroidal structure. The nozzle may be for outputting the particle beam to the treatment position. The nozzle may be mounted to the rotatable structure.

The gantry may include one or more imaging devices configured for movement around the toroidal structure and a nozzle that is configured for movement around the toroidal structure. The nozzle may be for outputting the particle beam to the treatment position. The nozzle and the one or more imaging devices may be mounted to the rotatable structure. The first magnets may be spaced apart and may each be located in a different circumferential sector of the toroidal structure. Each of the sectors may include a nozzle for outputting the particle beam to the treatment position.

Any two or more of the features described in this specification, including in this summary section, may be combined to form implementations not specifically described in this specification.

Control of the various devices, systems, and/or components described herein, or portions thereof, may be implemented via a computer program product that includes instructions that are stored on one or more non-transitory machine-readable storage media and that are executable on one or more processing devices (e.g., microprocessor(s), application-specific integrated circuit(s), programmed logic such as field programmable gate array(s), or the like). The devices, systems, and/or components described herein, or portions thereof, may be implemented as an apparatus, method, or electronic system that may include one or more processing devices and computer memory to store executable instructions to implement control of the stated functions. The devices, systems, and/or components described herein may be configured, for example through design, construction, arrangement, placement, programming, operation, activation, deactivation, and/or control.

The details of one or more implementations are set forth in the accompanying drawings and the following description. Other features and advantages will be apparent from the description and drawings, and from the claims.

### DESCRIPTION OF THE DRAWINGS

FIG. 1 is a diagram showing a partially cut-away, partly-transparent, side view of an example particle therapy system having an example toroidal gantry of the type described herein.

FIG. 2 is a diagram showing a partially-transparent, top view of the example particle therapy system of FIG. 1.

FIG. 3 is a diagram of a partially-transparent, perspective view showing components of the example particle therapy system of FIGS. 1 and 2.

FIG. 4 is a front view of part of an example toroidal gantry and sectors thereof.

FIG. 5 is a perspective diagram showing movement, at different times, of particle beams from a vector magnet to different bending magnets and then from the bending magnets toward a common treatment position at different angles.

FIG. 6 is a block diagram of components of an example nozzle that is configured to attach to an example toroidal gantry of the type described herein.

FIG. 7 is a perspective view of an example energy degrader that may be contained within the nozzle of FIG. 6.

FIG. 8 is a block diagram of an example treatment space that is configured to house the particle therapy system of FIGS. 1 to 3.

FIG. 9 is a perspective view of an example patient couch that may be used in the particle therapy system of FIGS. 1 to 3.

FIG. 10 is a diagram of a partially-transparent, perspective view showing components of the example particle therapy system of FIGS. 1 and 2.

FIG. 11 is a cut-away, side view of components in an example particle accelerator that may be used with the particle therapy system described herein.

Like reference numerals in different figures indicate like elements.

### DETAILED DESCRIPTION

Described herein are example particle therapy systems that may be housed in the same space that is used for treatment. Such a system includes a particle accelerator that may be, but is not limited to, a synchrocyclotron that is lightweight and that is small enough to fit within a standard linear accelerator (LINAC) vault. The system also includes a medical gantry configured to deliver a charged particle beam, such as protons or ions output from the accelerator, to treat tumors or other conditions in a patient. In this example, the gantry includes a toroidal (e.g., donut-shaped) structure having magnets in an interior thereof. The magnets include a first/vector magnet proximate to an output of the particle accelerator and second/bending magnets proximate to a treatment position. The treatment position may correspond to a location of a common isocenter for the bending magnets. The vector magnet is configured to direct the particle beam to different bending magnets, and the bending magnets are configured to bend the particle beam towards the treatment position. To enable delivery of the particle beam in the same space that is used for treatment, particularly in relatively small spaces such as a standard LINAC vault, the bending magnets are configured to bend the particle beam at right angles or at obtuse angles. For example, each of the bending magnets may be “D”-shaped and each of the bending magnets may be configured and arranged to bend the particle beam by 90° or greater.

In this regard, some implementations of the particle therapy system described herein employ large-aperture superconducting bending magnets that are configured to bend the particle beam within a short distance, thereby reducing the size of the system. In general, a superconductor is an element or metallic alloy such as niobium-tin (Nb₃Sn) which, when cooled below a threshold temperature, loses most, if not all, electrical resistance. As a result, current flows through the superconductor substantially unimpeded. Superconducting coils, therefore, are capable of conducting much larger currents in their superconducting state than ordinary wires of the same size. Because of the high amounts of current that they are capable of conducting, superconducting coils are particularly useful in particle therapy systems.

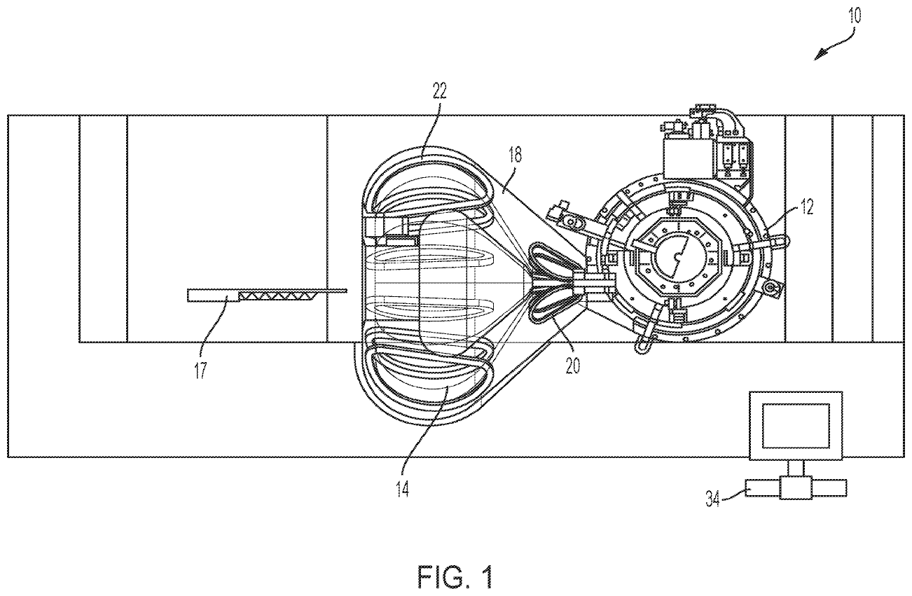

FIGS. 1, 2, and 3, show side, top, and perspective views, respectively, of the same particle therapy system 10. Particle therapy system 10 is of the type described in the preceding paragraphs. As shown, particle therapy system 10 includes a particle accelerator 12, examples of which are described herein. In this example, particle accelerator 12 is a synchrocyclotron having a superconducting electromagnetic structure that generates a maximum magnet field strength of 3 Tesla (T) or more. The example synchrocyclotron produces a particle beam having an energy level of 150 megaelectronvolts (MeV) or more, has a volume of 4.5 cubic meters (m³) or less, and has a weight of 30 Tons (T) or less. However, synchrocyclotrons or other types of particle accelerators having weights, dimensions, magnetic fields, and/or energy levels other than these may be used in particle therapy system 10.

Particle therapy system 10 also includes gantry 14. Gantry 14 is at least partially toroidal in structure as shown in FIGS. 1, 2, and 3. A toroid includes a donut-shape that is formed from surface generated by a closed-plane curve rotated about a line that lies in the same plane as the closed-plane curve. For example, a toroid may be defined parametrically by the following or similar equation set:

$$x ( u , v ) = ( cos u ) ( a cos ( v ) + c ) \qquad ( 1 )$$

$$y ( u , v ) = ( sin u ) ( a cos ( v ) + c ) \qquad ( 2 )$$

$$z ( u , v ) = a sin v \qquad ( 3 )$$

for a torus with having center at an origin, a rotational axis of symmetry about a z-axis, a radius c from the center of a hole to a center of a torus tube or opening, and radius a of the tube or opening. In this example, gantry 14 has a generally toroidal shape, as shown in FIG. 1, but need not strictly confirm to the mathematical definition of a toroid.

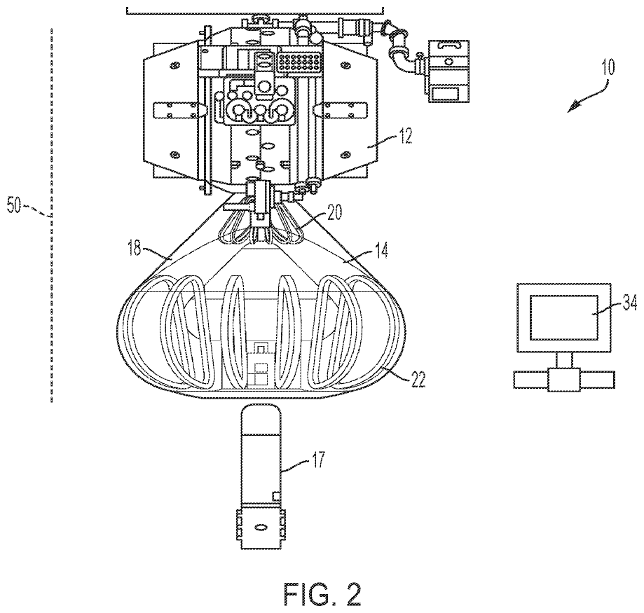

Gantry 14 thus includes a hole 15 through which treatment couch 17 is movable to place the patient in a treatment position. The interior of the toroidal structure of gantry 14 is defined by enclosure 18 (FIGS. 1 and 2). Within that interior is a vector magnet 20 (FIGS. 1 and 2), which is also referred to as a kicker magnet. In some implementations, gantry 14 may include more than one vector magnet. Vector magnet(s) 20 is/are configured and controllable to direct a particle bream originating at, and received from, particle accelerator 12 towards bending magnets 22 (FIGS. 1 and 2). In operation, vector magnet(s) 20 is/are configured to direct the particle bream received from the particle accelerator towards one of bending magnets 22 and to redirect the particle beam to a different bending magnet 22. For example, vector magnet(s) 20 is/are controllable to steer the particle beam between or among different ones of the bending magnets 22 in accordance with specifications of a treatment plan. As described below, the bending magnets are configured to direct the particle beam towards treatment position. Accordingly, by steering the particle beam between or among the bending magnets 22, vector magnet(s) 20 is/are able to control the direction from which the particle beam reaches an irradiation target at the treatment position. For example, by steering the particle beam between or among the bending magnets 22, vector magnet(s) 20 is/are able to control the different angles from which the particle beam is applied to an irradiation target at the treatment position.

In some implementations, there are multiple vector magnets, which may include dipole magnets configured and controllable to switch the particle beam among multiple paths. For example, each vector magnet may be configured to direct the particle beam to a single bending magnet or to a unique set of bending magnets. In operation, each vector magnet is controllable to switch-on rapidly and then to maintain a stable magnetic field for a predefined time. The remaining vector magnets may be switched-off at this time, enabling the switched-on vector magnet to direct the particle beam towards a target bending magnet. Different vectors magnets may be switched-on and/or switched-off to control the direction of the particle beam.

In some implementations, the vector magnet(s) are controllable in two dimensions (e.g., Cartesian XY dimensions a plane) to direct the particle beam towards a bending magnet. In some implementations, a vector magnet includes a first set of two coils, which control particle beam movement in the Cartesian X dimension, and a second set of two coils, which are orthogonal to the first set of two coils and which control particle beam movement in the Cartesian Y dimension. Control is achieved, in some implementations, by varying current through one or both sets of coils to thereby vary the magnetic field(s) produced thereby. By varying the magnetic field(s) appropriately, the particle beam can be moved in the X dimension and/or the Y dimension towards a bending magnet. For example, the X and Y dimensions correspond to a plane that is parallel to the radius of the toroidal structure and the vector magnet(s) 20 may direct the particle beam anywhere in that plane.

As noted, in some implementations, gantry 14 includes an enclosure 18 that physically connects to particle accelerator 12. Enclosure 18 may surround and enclose vector magnet(s) 20 and the particle beam(s) directed by vector magnet(s) 20. Enclosure 18 may also surround and enclose bending magnets 22 and other system components described herein including, but not limited to, nozzles and their internal components and imaging devices. Enclosure 18 may be electromagnetically shielded using materials such as lead, borated polyethylene, and/or steel.

As shown in FIGS. 1 to 3, bending magnets 22 are arranged circumferentially around hole 15 through the gantry's toroidal structure. Bending magnets 22 are substantially D-shaped in this example; however, magnets having other shapes may be used in place of D-shaped magnets. In this example, all of bending magnets 22 have the same shape; however, in other implementations, there may be differently-shaped bending magnets 22 occupying different sectors of gantry 14.

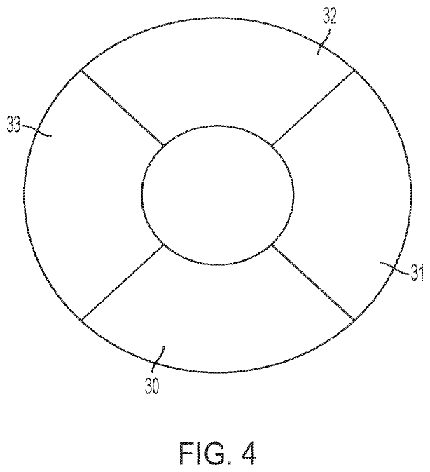

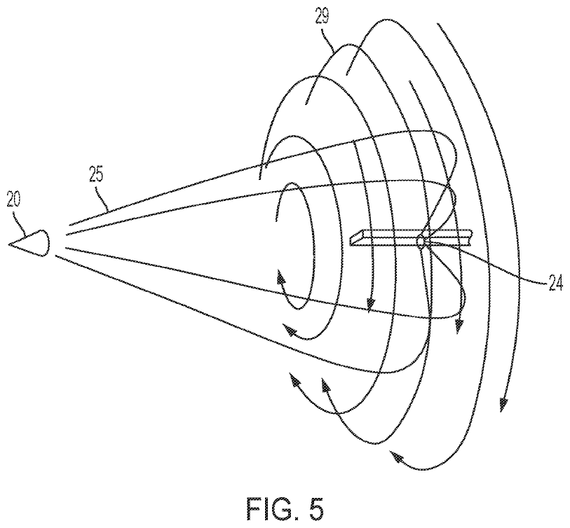

In this regard, as previously noted, gantry 14 is multi-sectored. FIG. 4 shows, conceptually an example toroidal gantry having four sector 30 to 33, each containing a bending magnet 22 of the type described herein. Other implementations such as that shown in FIGS. 1 and 2, may include more than four sectors or fewer than four sectors, each containing a corresponding bending magnet. Due to the positioning and shape of a bending magnet contained in each sector, each of the sectors is configured to deliver radiation to a patient from a different angular position. In this regard, in some implementations, the D-shape of each bending magnet 22 creates a magnetic field that bends the particle beam from accelerator 12 by 90° towards a treatment position 24. An example of this bending is shown, for example, in FIG. 5.

As show in FIG. 5, vector magnet 20 directs particle beams 25 at different times to different ones of the bending magnets 22 (the bending magnets are not shown in FIG. 5). Bending magnets 22 bend the particle beams 25 towards the treatment position 24. In some implementations, each bending magnet 22 or sector of gantry 14 may be configured—for example, shaped—to bend particle beam within a range of 90° to 150° or more—for example, 90°, 95°, 100°, 105°, 110°, 115°, 120°, 125°, 130°, 135°, 140°, 145°, or 150°. In some implementations, each bending magnet 22 or sector of gantry 14 may be configured—for example, shaped—to bend particle beam by more than 150° or less than 90°. Because the sectors and bending magnets are at different angular positions around the gantry, the particle beams applied via different bending magnets hit the irradiation target from different angles and, in some cases, at different locations. Lines 29 in FIG. 5 represent the magnetic field in the toroidal structure.

In some implementations, bending magnets 22 may be large-aperture superconducting magnets; however, non-superconducting magnets may be used alone or in combination with superconducting magnets. In some implementations there may be between 8 and 12 bending magnets 22 arrange circumferentially around hole 15. In some implementations there may be between 6 and 20 bending magnets 22 arranged circumferentially around hole 15. The separation distance between adjacent bending magnets may be the same for all bending magnets; that is, they may be spaced at uniform intervals around the toroidal gantry. The more bending magnets 22 that are located within gantry 14, the greater the gantry's precision may be. In this regard, in some implementations, gantry 14 remains stationary and does not rotate relative to the treatment position. In some implementations, gantry 14 may rotate to reach a desired angular position, but then not rotate during treatment. For example, in some implementations, gantry 14 includes one or more motors that enable its angular rotation by 180° or more, which enables precise angular placement of the bending magnets relative to the treatment position—for example, at 1° or less increments.

In either of the preceding cases, bending magnets 22 may remain stationary relative to the treatment position during treatment. Treatment may be implemented by moving the particle beam among different ones of bending magnets 22 in order to reposition the particle beam relative to an irradiation target at the treatment position. The more bending magnets 22 there are, the more accurately the particle beam can be positioned relative to the irradiation target. Stated differently, additional magnets provide for finer angular positioning of the particle beam relative to the irradiation target. The treatment angles may be set in a treatment plan based the physical constraints of the gantry, such as the number of available gantry output angles (which will be a function of the number of bending magnets 22), among other things.

In some implementations, individual ones of the bending magnets are controllable to move within the gantry. For example, motors attached to the magnets may control internal movement of the bending magnets to change the beam output. For example, moving the bending magnets within the gantry towards or away from the accelerator may affect the angle that the bending magnets bend the particle beam. For example, rotating or tilting the bending magnets within the gantry may affect the angle that the bending magnets bend the particle beam. Magnet movement may be in accordance with requirements of a treatment plan.

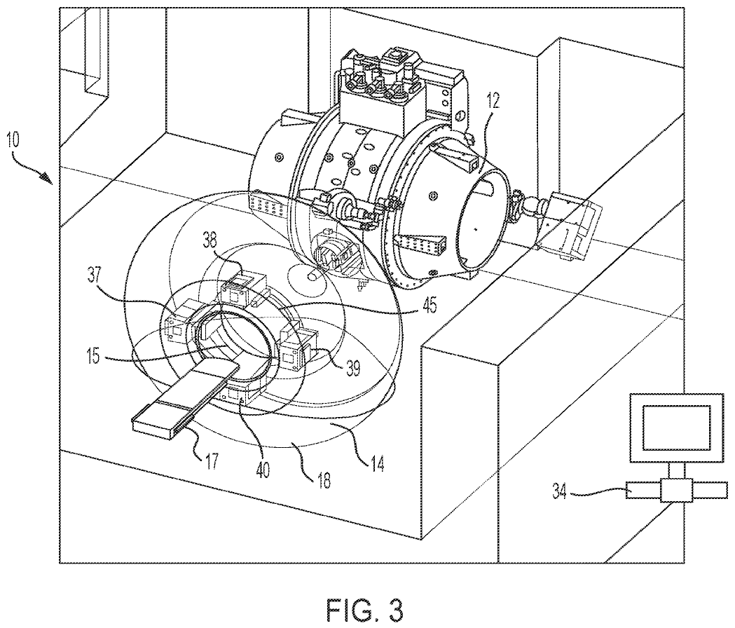

As explained, gantry 14 is multi-sectored. Due to the positioning and shape of the bending magnet contained in each sector, each of the sectors is configured to deliver radiation to a patient from a different angular position. A nozzle may be located in, or movable to, each sector. For example, in some implementations, gantry 14 includes a single nozzle that is mounted on a track or ring within enclosure 18 or on an external part of gantry 14. The nozzle is controllable using one more motors to move, during treatment, to a sector of the gantry and into the particle beam path output path of a bending magnet 22. A control system 34 such as those described herein may control movement of the nozzle along the gantry based on the treatment plan. In some implementations, the nozzle or multiple nozzles may be mounted on a ring 45 (FIG. 3) that is rotatable within gantry 14 and that is configured for mounting radiation delivery components such as the nozzle. The ring is rotatable to position the nozzles relative to the patient. The particle beam may be at its maximum energy as it passes over and beyond the bending magnets and enters the nozzle. In some implementations, the maximum energy may be the maximum beam energy that the accelerator is capable of outputting or a predefined beam energy set within the accelerator.

In some implementations, gantry 14 includes multiple nozzles that are mounted on one or more tracks or rings within gantry 14 or on the external part of gantry 14. For example, FIG. 3 shows four nozzles 37, 38, 39, and 40 mounted to gantry 14 on the interior of gantry 14. The nozzles 37, 38, 39, and 40 may be controlled using motors to move to a sector of the gantry and into the particle beam path output of a bending magnet 22. Alternatively, ring 45 may be rotated to angularly position the nozzles or the nozzles may be configured to move—for example, to rotate—around ring 45.

A control system such as those described herein may control movement of the nozzles based on the treatment plan. Each nozzle may be configured to serve one or more sectors of gantry 14. For example, a toroidal gantry may include twelve sectors and four nozzles. Each nozzle may be configured and controllable to service three adjacent sectors. For example, a first nozzle may service sectors one, two, and three; a second nozzle may service sectors four, five, and six; a third nozzle may service sectors seven, eight, and nine; and a fourth nozzle may service sectors ten, eleven, and twelve. Movement of the nozzles may be coordinated or limited to prevent collisions between nozzles during treatment. For example, the first nozzle may be configured to move in a range of 0° to 90° around the gantry, the second nozzle may be configured to move in a range of 91° to 180° around the gantry; the third nozzle may be configured to move in a range of 181° to 270° around the gantry; and the fourth nozzle may be controlled to move with a range of 279° to 359° around the gantry.

As described herein, in the toroidal gantry architecture, a vector magnet is located at or near the accelerator exit port and can change the beam angle rapidly resulting in significantly shorter total treatment times by delivering radiation from multiple angles simultaneously on a layer by layer or pulse by pulse basis. For this purpose, multiple nozzles (for example, 2, 3, 4 or more nozzles) can be mounted on rotatable ring 45 at an inner diameter of gantry 14. Because the ring rotates, one or more of the nozzles mounted thereto rotate with the ring, enabling positioning of the nozzles while reducing the chances of collisions with the patient or system components.

In some implementations, nozzles on gantry 14 are stationary; that is, the nozzles do not move relative to gantry 14. For example, there may be one nozzle in each sector that is in the beam path output of a bending magnet in that sector. Including one nozzle per sector with no movement may reduce treatment time, since no time is required to position and reposition the nozzles. In some implementations, all components mounted to gantry 14 do not move rotationally around the gantry. In addition, as noted, in some implementations gantry 14 itself is stationary. In addition, as noted, in some implementations gantry 14 itself is rotatable.

In the example of FIG. 3, nozzle 39 is located in a sector. In some implementations, each nozzle may have the same function and configuration. Nozzle 39 receives the particle beam from a bending magnet 22 and, in some implementations, conditions the particle beam for output to an irradiation target at the treatment position, such as a tumor in a patient. In this regard, as noted, each bending magnet 22 bends the particle beam by at least 90° toward a patient at the treatment position. The particle beam is thus directed towards the treatment position after it bends over a magnet 22.

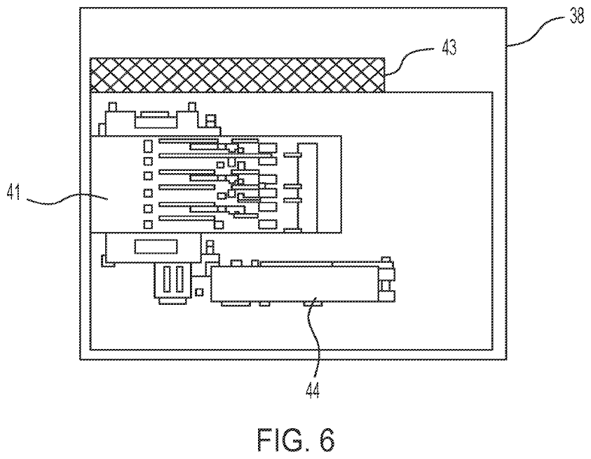

In some implementations, each bending magnet may include a scanning magnet at its output or incorporate scanning magnet functionality into its magnetic structure. A scanning magnet is controllable in two dimensions (e.g., Cartesian XY dimensions) to position the particle beam in those two dimensions and to move the particle beam across at least a part of an irradiation target. In some implementations, a scanning magnet includes a first set of two coils, which control particle beam movement in the Cartesian X dimension, and a second set of two coils, which are orthogonal to the first set of two coils and which control particle beam movement in the Cartesian Y dimension. Control is achieved, in some implementations, by varying current through one or both sets of coils to thereby vary the magnetic field(s) produced thereby. By varying the magnetic field(s) appropriately, the particle beam can be moved in the X and/or Y dimension across layers of the irradiation target. In some implementations, one or more scanning magnets may be downstream of—that is, closer to the treatment position than—each bending magnet but upstream of the energy degrader. For example, one or more scanning magnets 43 (FIG. 6) may be located in each nozzle between the energy degrader described below and the output of a bending magnet.

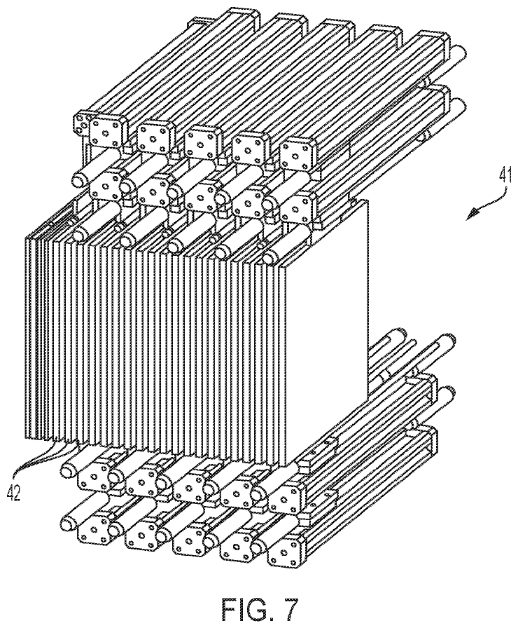

Referring to FIGS. 6 and 7, each nozzle, such as nozzle 38, may also include an energy degrader 41 that receives the particle beam from a bending and/or scanning magnet before the particle beam reaches the patient. In this example, energy degrader 41 is between a bending magnet 22 and the irradiation target. Energy degrader 41 is configured to, and controllable to, change an energy of the particle beam before the particle beam reaches the irradiation target. For example, the energy degrader may include plates that are movable into or out of a path of the particle beam. For example, the energy degrader may include wedges that overlap at least in part and that are movable within a path of the particle beam. An example wedge is a polyhedron defined by two triangles and three trapezoidal faces. In either configuration, variable amounts of material are movable into the path of the particle beam. The material absorbs energy from the particle beam, resulting in reduced-energy beam output. The more material there is in the path of the particle beam, the less energy that the particle beam will have. In some implementations, the energy-absorbing structures are movable across all of the beam field or across only part of the beam field over which the scanning magnet scans or over which the particle beam can be delivered. In some examples, the beam field is the maximum extent that the particle beam can be moved across a plane parallel to the treatment area on a patient for a given bending magnet or scanning magnet.

In the example of FIG. 7, energy degrader 41 is a range shifter that is controllable to move structures 42 into, and out of, the path of the particle beam to change the energy of the particle beam and therefore the depth to which dose of the particle beam will be deposited in the irradiation target. Examples of such structures include, but are not limited to, energy-absorbing plates; polyhedra such as wedges, tetrahedra, or toroidal polyhedra; and curved three-dimensional shapes, such as cylinders, spheres, or cones. In this way, the energy degrader can cause the particle beam to deposit doses of radiation in the interior of an irradiation target to treat layers or columns of the target. In this regard, when protons move through tissue, the protons ionize atoms of the tissue and deposit a dose along their path. The energy degrader thus is configured to move the particle beam in the Cartesian Z dimension through the target, thereby enabling scanning in three dimensions. In some implementations, the energy degrader may be configured to move during movement of the particle beam and to track or to trail the particle beam during movement. An example energy degrader that tracks or trails particle beam movement is described in U.S. Pat. No. 10,675,487 (Zwart) entitled “High-Speed Energy Switching”. The content of U.S. Pat. No. 10,675,487, particularly the content related to the energy degrader that tracks or trails particle beam movement (e.g., FIGS. 36 to 46 of U.S. Pat. No. 10,675,487 and the accompanying description), is incorporated herein by reference.

The Bragg peak is a pronounced peak on the Bragg curve that plots the energy loss of ionizing radiation during its travel through tissue. The Bragg peak represents the depth at which most protons deposit within tissue. For protons, the Bragg peak occurs right before the particles come to rest. Accordingly, the energy of the particle beam may be changed to change the location of its Bragg peak and, therefore, where a majority of the dose of protons will deposit in depth in the tissue. In this regard, in some implementations, the particle accelerator is a fixed-energy particle accelerator. In a fixed-energy particle accelerator, the particle beam always exits the particle accelerator at the same, or about the same, energy—for example, within a 5% deviation or less from an expected or target energy. In a fixed-energy particle accelerator, the energy degrader is the primary vehicle for varying the energy of the beam applied to an irradiation target in the patient. In some implementations, the particle accelerators described herein are configured to output particle beams at a single (fixed) energy or at two or more energies within a range between about 100 MeV and about 300 MeV (for example, between 115 MeV and 250 MeV). The fixed energy output may be within that range (e.g., 250 MeV) or, in some examples, above or below that range.

In some implementations, the particle accelerator is a dual-energy accelerator. In a dual-energy particle accelerator, the particle beam exits the particle accelerator at one of two different energy levels—a high energy level and a low energy level. The terms “high” and “low” have not specific numerical connotations but rather are intended to convey relative magnitudes. In some implementations, the particle accelerators described herein are configured to output particle beams at two energies that are within a range that is between about 100 MeV and about 300 MeV. The high energy output and the low energy output may be values within that range or, in some examples, above or below that range. The energy degrader described herein may be used with dual-energy particle accelerators in order to reduce the energy of the particle beam below one of the two energy levels and/or to finely adjust the two energy levels.

Nozzle 40 also includes a collimator 44 (FIG. 6) downstream of energy degrader 41 relative to the irradiation target (that is, between the energy degrader and the target). In an example, a collimator is a device that is controllable to allow some radiation to pass to a patient and to block some radiation from passing to the patient. Typically, the radiation that passes is directed to an irradiation target to be treated, and the radiation that is blocked would otherwise hit, and potentially damage, healthy tissue. In operation, the collimator is placed in the radiation path between a bending magnet and the irradiation target and is controlled to produce an opening of an appropriate size and shape to allow some radiation to pass through the opening to the irradiation target, while a remainder of the structure blocks some radiation from reaching adjacent tissue. An example of a configurable collimator that may be used is described in U.S. Patent Publication No. 2017/0128746 (Zwart) entitled “Adaptive Aperture”. The content of U.S. Patent Publication No. 2017/0128746, particularly the content relating to the description of the adaptive aperture (e.g., FIGS. 1 to 7 of U.S. Patent Publication No. 2017/0128746 and the accompanying description), is incorporated herein by reference.

As described herein, an example particle therapy system includes an example toroidal gantry that utilizes several large aperture superconducting magnets. In this type of gantry, the source-at-distance (SAD) satisfies clinical requirements and the overall diameter of the gantry may be reduced to less than 5 meters (m). In an example, the overall diameter of gantry 14 is 3.2 m for a 250 MeV implementation. In this example, the total length 50 (FIG. 2) of gantry 14 is less than 5 m, e.g., approximately 4.3 m, and the distance between the output of each bending magnet 22 to the treatment position is 1 m or less. In this regard, in some implementations, the distance between the output of each bending magnet 22 and the treatment position is 2 m or less. In some implementations, the distance between the output of each bending magnet 22 and the treatment position is 1 m or less. In some implementations, the distance between the output of each bending magnet 22 and the treatment position is 0.5 m or less. In some implementations, the gantry weights 17 tons or less.

Other implementations may have different dimensions including, but not limited to, the gantry diameters and distances noted here. In some implementations, the particle therapy system can fit within the footprint of LINAC vault. For example, the components of FIGS. 1 to 6 may be small enough fit within, and have dimensions that fit within, a vault having the following dimensions: 25 feet (7.62 meters (m)) or less in length, 20 feet (6.09 m) or less in width, and 11 feet (3.35 m) or less in height. For example, the components of FIGS. 1 to 6 may be small enough fit within, and have dimensions that fit within, a vault having the following dimensions: 25 feet (7.62 meters (m)) or less in length, 26 feet (7.92 m) or less in width, and 10 feet (3.05 m) or less in height. For example, the components of FIGS. 1 to 3 may be small enough fit within, and have dimensions that fit within, a LINAC vault having a footprint of 26.09 feet (11 m) by 29.62 feet (9 m) or less, with a height of 16.40 feet (5 m) or less. However, as noted, some implementations of the particle therapy system may have different dimensions including, but not limited to, diameters, lengths, widths, and/or heights. In some implementations, the ceiling of a pre-existing LINAC vault may not be high enough to support the full diameter of the gantry. In such implementations, a pit may be dug beneath the floor of the LINAC vault to enable the gantry to fit within the vault.

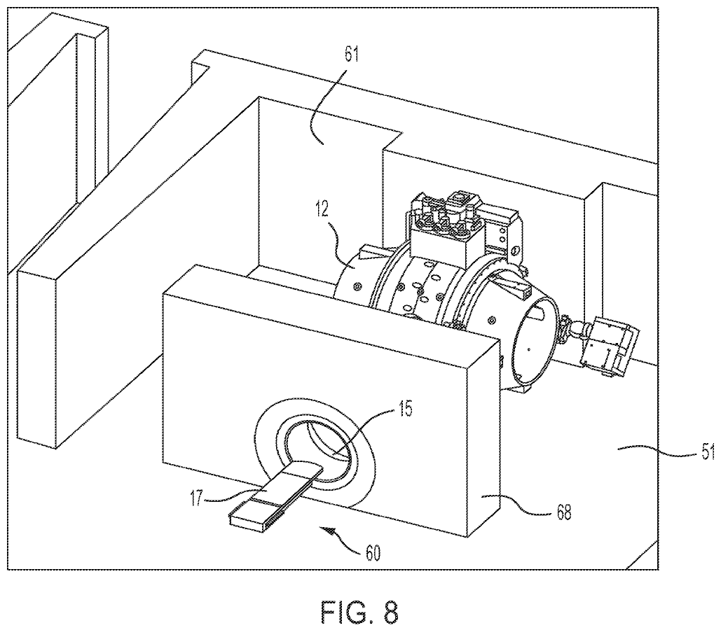

FIG. 8 shows an example of a treatment space 51 in which particle therapy system 10 and its variants may be implemented. The treatment space is implemented in a LINAC vault in this example and is shielded using lead or other appropriate materials such as such as concrete, borated polyethylene, and/or steel. In this regard, particles, such as protons, that are created by the particle accelerator but do not reach the irradiation target create secondary radiation through the production of high energy neutrons. In some implementations, the gantry described herein, which includes energy selection using an energy degrader that is downstream of the beamline, is configured to transmit more than 70% of the proton beam even at low energies. In this regard, in some implementations, the particle beam is at the maximum energy and fixed energy of the accelerator until just upstream of isocenter (at the inner diameter of the toroid) where it is reduced in energy by a dynamic range shifter. That is, the particle beam is at the maximum energy of the accelerator as the particle beam enters the nozzle. Due to the high beam delivery efficiency (e.g., 70 to 100%) of this direct beam system architecture, which maintains a low stray radiation from the accelerator and energy modulation, the accelerator can be located within the treatment room vault as shown in FIG. 8. That is, the direct beam architecture described herein enables high efficiency transfer of the particle beam, which reduces stray radiation. Because the stray radiation is reduced, the accelerator and patient can be located in the same treatment space without fear of the stray radiation harming the patient or electronic equipment.

In some implementations, the particle beam output by the accelerator may be monoenergetic and the energy degrader is the only/sole or primary vehicle for changing beam energy during treatment of an irradiation target. A monoenergetic particle beam includes a particle beam having a single, fixed energy level, such as 100 MeV, 150 Mev, 200 Mev, 250 Mev, and so forth. A monoenergetic particle beam may deviate from the fixed energy level by a predetermined amount, such as ±10%, ±5%, ±2%, or ±1%, and still be considered monoenergetic if its energy is not actively changed. Switching operation of the accelerator during treatment, as is required to switch particle beam energies during treatment, may produce excess stray neutrons, resulting in the need for increased shielding and reducing beamline efficiency. The neutrons may be generated by the particle accelerator and/or by magnetics along the beamline. By using a particle beam that is monoenergetic during treatment and relying on the energy degrader to change beam energy, production of stray neutrons may be reduced or minimized and the efficiency of the particle beam output may be increased.

Use of a monoenergetic particle beam, use of an energy degrader that is outside of the gantry, and use of a toroidal gantry having vector and bending magnets as described herein enables the particle beam to be directed efficiently. More specifically, changes in beam energy increase production of stray neutrons and, therefore, losses of particle beam, thereby degrading its efficiency. The monoenergetic particle beam used in the implementations of the systems described herein, combined with a toroidal gantry having vector and bending magnets, may lead to increased efficiency. In some cases, the direct beam architecture described herein produces an efficiency of 10% or more, 20% or more, 30% or more, 40% or more, 50% or more, 60% or more, 70% or more, 80% or more, or 90% or more. In some examples, efficiency is a measure of the percentage of particles output from the particle accelerator that are output from the bending magnets. So, an efficiency of 10% or more includes 10% or more of the particles output from the particle accelerator being output from the bending magnets; an efficiency of 20% or more includes 20% or more of the particles output from the particle accelerator being output from the bending magnets; an efficiency of 30% or more includes 30% or more of the particles output from the particle accelerator being output from the bending magnets; an efficiency of 40% or more includes 40% or more of the particles output from the particle accelerator being output from the bending magnets; an efficiency of 50% or more includes 50% or more of the particles output from the particle accelerator being output from the bending magnets; an efficiency of 60% or more includes 60% or more of the particles output from the particle accelerator being output from the bending magnets; an efficiency of 70% or more includes 70% or more of the particles output from the particle accelerator being output from the bending magnets; an efficiency of 80% or more includes 80% or more of the particles output from the particle accelerator being output from the bending magnets; and an efficiency of 90% or more includes 90% or more of the particles output from the particle accelerator being output from the bending magnets. In an example, the particle accelerator and gantry described herein transmit more than 70% of a proton beam to a patient even at energies in lower range of the accelerator. The direct beam architecture of the type described herein enables a “single room” solution in which the particle accelerator, the gantry, and patient all reside with a single room or vault.

In contrast, some particle therapy systems employ energy selection systems that result in significant production of high-energy neutrons and that discard more than 99% of the proton beam at lower energies. Beamline efficiency of the type described herein may enable a “single room” solution that puts the particle accelerator, beamline, and patient all inside a single vault. Within this vault, the particle accelerator may include shielding, but separate compartments 60 and 61 in the vault containing the patient and the particle accelerator, respectively, need not be shielded from each other. In other words, in some implementations, there is no shielding that is external to the particle accelerator and the gantry that separates the particle accelerator from the patient. That is, in some examples, the only shielding that separates the particle accelerator from the patient is within the particle accelerator itself or within the gantry itself. In FIG. 8, parts of gantry 14 are located within wall 58 separating compartments 60 and 61.

In some implementations, to be capable of installing a proton therapy system in an existing vault, the vault is capable of providing the necessary shielding, which may require that shielding be added. In the case of a toroidal gantry that does not rotate, shielding can be added local to the toroid along the beam plane. The shielding can be made of typical shielding materials such as concrete, borated polyethylene, and steel.

Referring to FIGS. 1, 2, 3, 8, and 9 particle therapy system 10 includes a treatment couch 17. Treatment couch 17 is configured to move relative to and within hole 15 in or through the toroidal gantry 14. In this example, treatment couch 17 may be mounted to a robotic arm 54. Arm 54 includes a first segment 55, a second segment 56, and third segment 57. First segment 55 is rotatably coupled to second segment 56 and second segment 56 is rotatably coupled to third segment 57. Treatment couch 17 is connected to third segment 57 as shown in the figure. Arm 54 is controllable to move treatment couch 17 in and through hole 15 to position a patient lying on the couch for treatment; that is, to move the patient into the treatment position.

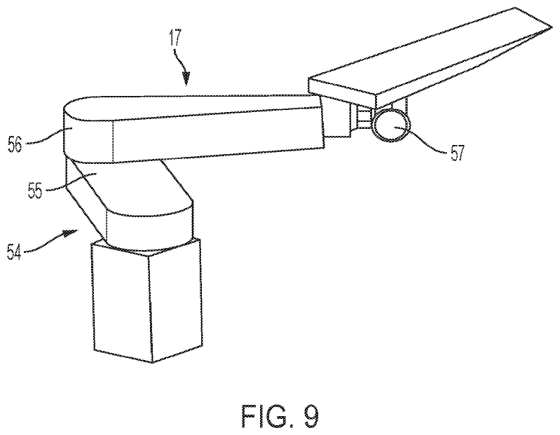

In some implementations, arm 54 may position the patient in two degrees of freedom, in three degrees of freedom, in four degrees of freedom, in five degrees of freedom, or in six degrees of freedom. An example of two degrees of freedom is forward-backward movement and left-right movement; an example of three degrees of freedom is forward-backward movement, left-right movement, and up-down movement; an example of four degrees of freedom is forward-backward movement, left-right movement, up-down movement and one of pitch, yaw, or roll movement; an example of five degrees of freedom is forward-backward movement, left-right movement, up-down movement and two of pitch, yaw, or roll movement; and an example of six degrees of freedom is forward-backward movement, left-right movement, up-down movement, pitch movement, yaw movement, and roll movement. In some implementations, the treatment couch may be replaced by a couch that inclines at least in part or by a chair, either of which may be controllable in two, three, four, five, or six degrees of freedom to position the patient for treatment. In some implementations, arm 54 may have a different configuration than that shown in FIG. 9. For example, arm 54 may have two segments or more than three segments. Hydraulics, robotics, or both, may control or implement non-planar movement of the treatment couch.

In some implementations, the treatment couch or other seat is configured to move relative to the particle beam during treatment. This is particularly true of systems in which the gantry does not move relative to the patient. In some implementations, treatment may be implemented using a combination of beam movement and treatment couch (or other seat movement). For example, the gantry may be positioned—for example, rotated—and the beam may be fixed temporarily, during which time the treatment couch moves to implement treatment. After that, the gantry may be repositioned to fix the beam temporarily at a new position. Treatment may be implemented at the new position through couch movement. These operations may be repeated as defined by a treatment plan drafted for use with the particle therapy system.

Particle therapy system 10 may be an intensity-modulated proton therapy (IMPT) system. IMPT systems enable spatial control of circumscribed beams of protons that may have a variable energy and/or intensity. IMPT takes advantage of the charged-particle Bragg peak—as noted, the characteristic peak of dose at the end of particles' delivery range—combined with the modulation of particle beam variables to create target-local modulations in dose to achieve objectives set forth in a predefined treatment plan. IMPT may involve directing particle beams toward the irradiation target at different angles and at different intensities to treat the target. This may be done by controlling the vector magnet to direct the particle beam to two or more different bending magnets. In some implementations, the particle beam may be scanned—for example, moved—across layers of the irradiation target, with each layer being treated one or more times from the same or different angles. Movement across the irradiation target to implement scanning may be performed using the scanning magnet(s) described herein.

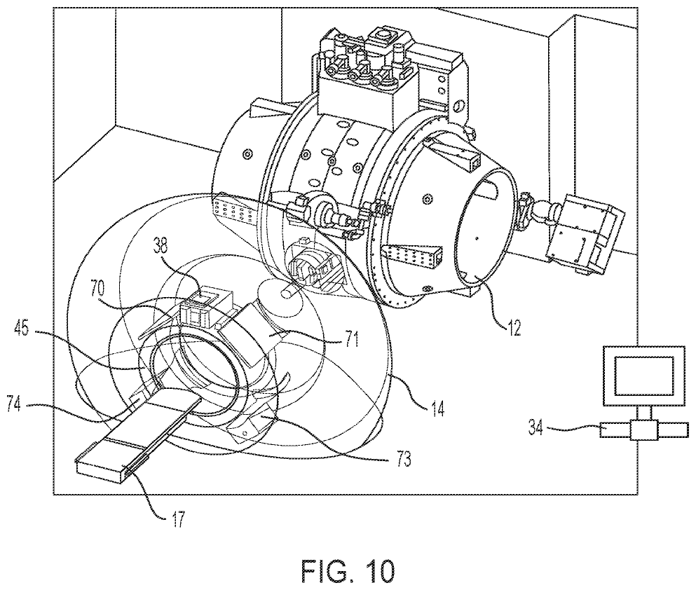

Referring to FIG. 10, one or more imaging devices may be mounted to gantry 14 within the interior of gantry 14 (as shown) or on the external part of gantry 14 (not shown). Imaging may be performed before and/or during treatment to identify a target location within the patient and to control operation of the gantry and scanning in order to direct the particle beam to the irradiation target in the patient.

The imaging devices may include, but are not limited to, one or more computed tomography (CT) systems, one or more fan-beam CT systems, one or more radiograph systems, and the like. The imaging system(s) may be configured and controlled to rotate around gantry 14 or to rotate along with rotation of gantry 14. In this regard, as noted one or more nozzles are rotatable on a ring 45 located at the gantry's inner diameter. A variety of two-dimensional (2D) and/or three-dimensional (3D) imaging devices also may be mounted on the ring 45 to be rotatable therewith. In some implementations, the nozzles and imaging devices may be mounted to different internal circumferential tracks within the gantry. For example, nozzles may be rotatable around a circumferential track at a first radius of the toroidal structure, and imaging devices may be rotatable around a different circumferential track at a second radius of the toroidal structure that is different from the first radius. In some implementations, the gantry may include different rotatable inner rings, one of which mounts the nozzles for rotation and one of which mounts the imaging devices or systems for rotation.

In the example of FIG. 10, two planes 70, 71 of orthogonal 2D imaging are shown for use with 2D image-guided radiation therapy (IGRT) or that can be rotated for cone-beam computed tomography systems (CBCT) acquisition including simultaneously acquired dual energy imaging. In this regard, IGRT includes the use of imaging during radiation treatment to improve the precision and accuracy of treatment delivery. IGRT may be used to treat tumors in areas of the body that move, such as the lungs. The imaging devices may also, or alternatively, include an X-ray source and an image panel for CBCT acquisition device 73 or a fan-beam diagnostic quality computed tomography (CT) device 74. Alternatively, one plane may include a CBCT and another plane may include fan-beam diagnostic quality CT.

In some implementation, control system 34 may coordinate rotation of nozzle 38, and of the imaging systems 70, 71, 73, 74 so that the imaging systems are not in the way of the nozzle and output channel and during treatment, and so that the nozzle and output channel are not in the way of the imaging system(s) during treatment. In some implementations, the imaging system(s) may be mounted to separate track(s) on gantry 14 than the nozzles. Separate mounting may reduce the chances of interference or collision between the nozzles and the imaging devices or systems. In some implementations, the imaging system(s) may be mounted the same rotatable ring 45 as the nozzles. Such mounting may reduce the chances of interference or collision between the nozzles and the imaging devices or systems.

As explained previously, some of each of the nozzle(s) and imaging device(s) or system(s) described herein may be within the enclosure of the toroidal gantry. Mounting within the enclosure may enable rotation of those devices at greater rates than external mounting. This is because there is less danger to the patient and surrounding equipment when devices are internally mounted than when those devices are externally mounted. For example, some externally-mounted devices are limited to rotational speeds of one (1) rotation-per-minute (RPM). Internally-mounted components, however, may be rotated at greater rates such as up to 240 RPM.

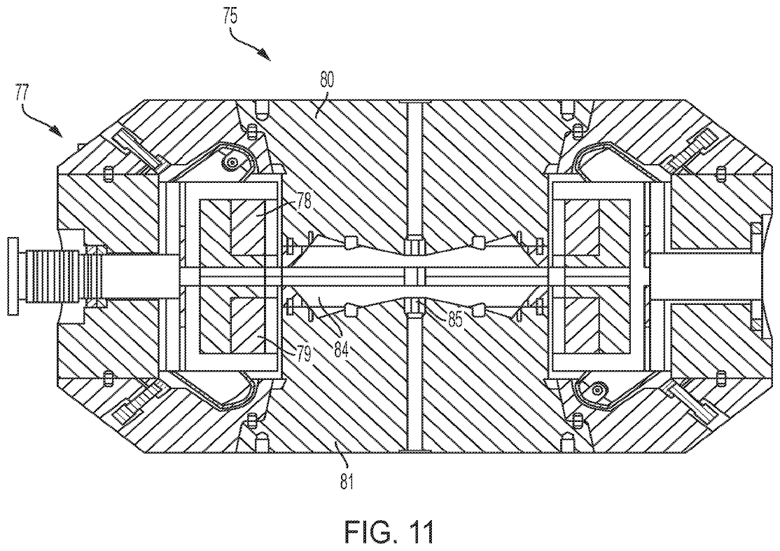

As described herein, an example proton therapy system scans a proton beam in three dimensions across an irradiation target in order to destroy malignant tissue. FIG. 11 shows a cross-section of components 75 of an example superconducting synchrocyclotron that may be used to provide the proton beam in the proton therapy system. In this example, components 75 include a superconducting magnet 77. The superconducting magnet includes superconducting coils 78 and 79. The superconducting coils are formed of multiple integrated conductors, each of which includes superconducting strands—for example, four strands or six strands—wound around a center strand which may itself be superconducting or non-superconducting. Each of the superconducting coils 78, 79 is for conducting a current that generates a magnetic field (B). The magnetic yokes 80, 81 or smaller magnetic pole pieces shape that magnetic field in a cavity 84 in which particles are accelerated. In an example, a cryostat (not shown) uses liquid helium (He) to conductively cool each coil to superconducting temperatures, e.g., around 4° Kelvin (K).

In some implementations, the particle accelerator includes a particle source 85, such as a Penning Ion Gauge—PIG source, to provide an ionized plasma column to cavity 84. Hydrogen gas, or a combination of hydrogen gas and a noble gas, is ionized to produce the plasma column. A voltage source provides a varying radio frequency (RF) voltage to cavity 84 to accelerate particles from the plasma column within the cavity. As noted, in an example, the particle accelerator is a synchrocyclotron. Accordingly, the RF voltage is swept across a range of frequencies to account for relativistic effects on the particles, such as increasing particle mass, when accelerating particles within the acceleration cavity. The RF voltage drives a dee plate contained within the cavity and has a frequency that is swept downward during the accelerating cycle to account for the increasing relativistic mass of the protons and the decreasing magnetic field. A dummy dee plate acts as a ground reference for the dee plate. The magnetic field produced by running current through the superconducting coils, together with sweeping RF voltage, causes particles from the plasma column to accelerate orbitally within the cavity and to increase in energy as a number of turns increases.

The magnetic field in the cavity is shaped to cause particles to move orbitally within the cavity. The example synchrocyclotron employs a magnetic field that is uniform in rotation angle and falls off in strength with increasing radius. In some implementations, the maximum magnetic field produced by the superconducting (main) coils may be within the range of 3 Tesla (T) to 20 T at a center of the cavity, which falls off with increasing radius. For example, the superconducting coils may be used in generating magnetic fields at, or that exceed, one or more of the following magnitudes: 3.0 T, 3.1 T, 3.2 T, 3.3 T, 3.4 T, 3.5 T, 3.6 T, 3.7 T, 3.8 T, 3.9 T, 4.0 T, 4.1 T, 4.2 T, 4.3 T, 4.4 T, 4.5 T, 4.6 T, 4.7 T, 4.8 T, 4.9 T, 5.0 T, 5.1 T, 5.2 T, 5.3 T, 5.4 T, 5.5 T, 5.6 T, 5.7 T, 5.8 T, 5.9 T, 6.0 T, 6.1 T, 6.2 T, 6.3 T, 6.4 T, 6.5 T, 6.6 T, 6.7 T, 6.8 T, 6.9 T, 7.0 T, 7.1 T, 7.2 T, 7.3 T, 7.4 T, 7.5 T, 7.6 T, 7.7 T, 7.8 T, 7.9 T, 8.0 T, 8.1 T, 8.2 T, 8.3 T, 8.4 T, 8.5 T, 8.6 T, 8.7 T, 8.8 T, 8.9 T, 9.0 T, 9.1 T, 9.2 T, 9.3 T, 9.4 T, 9.5 T, 9.6 T, 9.7 T, 9.8 T, 9.9 T, 10.0 T, 10.1 T, 10.2 T, 10.3 T, 10.4 T, 10.5 T, 10.6 T, 10.7 T, 10.8 T, 10.9 T, 11.0 T, 11.1 T, 11.2 T, 11.3 T, 11.4 T, 11.5 T, 11.6 T, 11.7 T, 11.8 T, 11.9 T, 12.0 T, 12.1 T, 12.2 T, 12.3 T, 12.4 T, 12.5 T, 12.6 T, 12.7 T, 12.8 T, 12.9 T, 13.0 T, 13.1 T, 13.2 T, 13.3 T, 13.4 T, 13.5 T, 13.6 T, 13.7 T, 13.8 T, 13.9 T, 14.0 T, 14.1 T, 14.2 T, 14.3 T, 14.4 T, 14.5 T, 14.6 T, 14.7 T, 14.8 T, 14.9 T, 15.0 T, 15.1 T, 15.2 T, 15.3 T, 15.4 T, 15.5 T, 15.6 T, 15.7 T, 15.8 T, 15.9 T, 16.0 T, 16.1 T, 16.2 T, 16.3 T, 16.4 T, 16.5 T, 16.6 T, 16.7 T, 16.8 T, 16.9 T, 17.0 T, 17.1 T, 17.2 T, 17.3 T, 17.4 T, 17.5 T, 17.6 T, 17.7 T, 17.8 T, 17.9 T, 18.0 T, 18.1 T, 18.2 T, 18.3 T, 18.4 T, 18.5 T, 18.6 T, 18.7 T, 18.8 T, 18.9 T, 19.0 T, 19.1 T, 19.2 T, 19.3 T, 19.4 T, 19.5 T, 19.6 T, 19.7 T, 19.8 T, 19.9 T, 20.0 T, 20.1 T, 20.2 T, 20.3 T, 20.4 T, 20.5 T, 20.6 T, 20.7 T, 20.8 T, 20.9 T, or more. Furthermore, the superconducting coils may be used in generating magnetic fields that are outside the range of 3 T to 20 T or that are within the range of 3 T to 20 T but that are not specifically listed herein.

In some implementations, such as the implementations shown in FIG. 11, the relatively large ferromagnetic magnetic yokes 80, 81 act as magnetic returns for stray magnetic fields produced by the superconducting coils. In some systems, a magnetic shield (not shown) surrounds the yokes. The return yokes and the shield together act to reduce stray magnetic fields, thereby reducing the possibility that stray magnetic fields will adversely affect the operation of the particle accelerator.

In some implementations, the return yokes and shield may be replaced by, or augmented by, an active return system. An example active return system includes one or more active return coils that conduct current in a direction opposite to current through the main superconducting coils. In some example implementations, there is an active return coil for each superconducting main coil, e.g., two active return coils-one for each main superconducting coil. Each active return coil may also be a superconducting coil that surrounds the outside of a corresponding main superconducting coil concentrically. By using an active return system, the relatively large ferromagnetic magnetic yokes 80, 81 can be replaced with magnetic pole pieces that are smaller and lighter. Accordingly, the size and weight of the synchrocyclotron can be reduced further without sacrificing performance. An example of an active return system that may be used is described in U.S. Pat. No. 8,791,656 (Zwart) entitled “Active Return System”. The content of U.S. Pat. No. 8,791,656, particularly the content related to the return coil configuration (e.g., FIGS. 2, 4, and 5 of U.S. Pat. No. 8,791,656 and the accompanying description), is incorporated herein by reference.

Another example of a particle accelerator that may be used in the particle therapy system herein is described in U.S. Pat. No. 8,975,836 (Bromberg) entitled “Ultra-Light Magnetically Shielded High-Current, Compact Cyclotron”. The content of U.S. Pat. No. 8,975,836, particularly the content related to “cyclotron 11” or “iron-free cyclotron 11” of FIGS. 4, 17 and 18 of U.S. Pat. No. 8,975,836 and the accompanying description, is incorporated herein by reference.

In some implementations, the synchrocyclotron used in the proton therapy system described herein may be a variable-energy synchrocyclotron. In some implementations, a variable-energy synchrocyclotron is configured to vary the energy of the output particle beam by varying the magnetic field in which the particle beam is accelerated. For example, the current may be set to any one of multiple values to produce a corresponding magnetic field. For example, the current may be set to one of two values to produce the dual-energy particle accelerator described previously. In an example implementation, one or more sets of superconducting coils receives variable electrical current to produce a variable magnetic field in the cavity. In some examples, one set of coils receives a fixed electrical current, while one or more other sets of coils receives a variable current so that the total current received by the coil sets varies. In some implementations, all sets of coils are superconducting. In some implementations, some sets of coils, such as the set for the fixed electrical current, are superconducting, while other sets of coils, such as the one or more sets for the variable current, are non-superconducting (e.g., copper) coils.

Generally, in a variable-energy synchrocyclotron, the magnitude of the magnetic field is scalable with the magnitude of the electrical current. Adjusting the total electric current of the coils in a predetermined range can generate a magnetic field that varies in a corresponding, predetermined range. In some examples, a continuous adjustment of the electrical current can lead to a continuous variation of the magnetic field and a continuous variation of the output beam energy. Alternatively, when the electrical current applied to the coils is adjusted in a non-continuous, step-wise manner, the magnetic field and the output beam energy also varies accordingly in a non-continuous (step-wise) manner. The step-wise adjustment can produce the dual energies described previously. In some implementations, each step is between 10 MeV and 80 MeV. The scaling of the magnetic field to the current can allow the variation of the beam energy to be carried out relatively precisely, thus reducing the need for an energy degrader. An example of a variable-energy synchrocyclotron that may be used in the particle therapy systems described herein is described in U.S. Pat. No. 9,730,308 entitled “Particle Accelerator That Produces Charged Particles Having Variable Energies”. The content U.S. Pat. No. 9,730,308 is incorporated herein by reference, particularly the content that enables operation of a synchrocyclotron at variable energies, including the content described in columns 5 through 7 of U.S. Pat. No. 9,730,308 and FIG. 13 and its accompanying description.

In implementations of the particle therapy system that use a variable-energy synchrocyclotron, controlling the energy of the particle beam to treat a portion of the irradiation target may be performed in accordance with the treatment plan by changing the energy of the particle beam output by the synchrocyclotron. In such implementations, a range shifter may or may not be used. For example, controlling the energy of the particle beam may include setting the current in the synchrocyclotron main coils to one of multiple values, each which corresponds to a different energy at which the particle beam is output from the synchrocyclotron. A range shifter may be used along with a variable-energy synchrocyclotron to provide additional changes in energy, for, example, between discrete energy levels provided by the synchrocyclotron.

The system and its variations described herein may be used to apply ultra-high dose rates of radiation—so called, “FLASH” dose rates of radiation—to an irradiation target in a patient. In this regard, experimental results in radiation therapy have shown an improvement in the condition of healthy tissue subjected to radiation when the treatment dose is delivered at ultra-high (FLASH) dose rates. In an example, when delivering doses of radiation at 10 to 20 Gray (Gy) in pulses of less than 500 milliseconds (ms) reaching effective dose rates of 20 to 100 Gray-per-second (Gy/S), healthy tissue experiences less damage than when irradiated with the same dose over a longer time scale, while tumors are treated with similar effectiveness. A theory that may explain this “FLASH effect” is based on the fact that radiation damage to tissue is proportionate to oxygen supply in the tissue. In healthy tissue, the ultra-high dose rate radicalizes the oxygen only once, as opposed to dose applications that radicalize the oxygen multiple times over a longer timescale. This may lead to less damage in the healthy tissue using the ultra-high dose rate.

In some examples, as noted above, ultra-high dose rates of radiation may include doses of radiation that exceed 1 Gray-per-second for a duration of less than 500 ms. In some examples, ultra-high dose rates of radiation may include doses of radiation that exceed 1 Gray-per-second for a duration that is between 10 ms and 5 s. In some examples, ultra-high dose rates of radiation may include doses of radiation that exceed 1 Gray-per-second for a duration that is less than 5 s.

In some examples, ultra-high dose rates of radiation include doses of radiation that exceed one of the following doses for a duration of less than 500 ms: 2 Gray-per-second, 3 Gray-per-second, 4 Gray-per-second, 5 Gray-per-second, 6 Gray-per-second, 7 Gray-per-second, 8 Gray-per-second, 9 Gray-per-second, 10 Gray-per-second, 11 Gray-per-second, 12 Gray-per-second, 13 Gray-per-second, 14 Gray-per-second, 15 Gray-per-second, 16 Gray-per-second, 17 Gray-per-second, 18 Gray-per-second, 19 Gray-per-second, 20 Gray-per-second, 30 Gray-per-second, 40 Gray-per-second, 50 Gray-per-second, 60 Gray-per-second, 70 Gray-per-second, 80 Gray-per-second, 90 Gray-per-second, or 100 Gray-per-second. In some examples, ultra-high dose rates of radiation include doses of radiation that exceed one of the following doses for a duration that is between 10 ms and 5 s: 2 Gray-per-second, 3 Gray-per-second, 4 Gray-per-second, 5 Gray-per-second, 6 Gray-per-second, 7 Gray-per-second, 8 Gray-per-second, 9 Gray-per-second, 10 Gray-per-second, 11 Gray-per-second, 12 Gray-per-second, 13 Gray-per-second, 14 Gray-per-second, 15 Gray-per-second, 16 Gray-per-second, 17 Gray-per-second, 18 Gray-per-second, 19 Gray-per-second, 20 Gray-per-second, 30 Gray-per-second, 40 Gray-per-second, 50 Gray-per-second, 60 Gray-per-second, 70 Gray-per-second, 80 Gray-per-second, 90 Gray-per-second, or 100 Gray-per-second. In some examples, ultra-high dose rates of radiation include doses of radiation that exceed one of the following doses for a duration that is less than 5 s: 2 Gray-per-second, 3 Gray-per-second, 4 Gray-per-second, 5 Gray-per-second, 6 Gray-per-second, 7 Gray-per-second, 8 Gray-per-second, 9 Gray-per-second, 10 Gray-per-second, 11 Gray-per-second, 12 Gray-per-second, 13 Gray-per-second, 14 Gray-per-second, 15 Gray-per-second, 16 Gray-per-second, 17 Gray-per-second, 18 Gray-per-second, 19 Gray-per-second, 20 Gray-per-second, 30 Gray-per-second, 40 Gray-per-second, 50 Gray-per-second, 60 Gray-per-second, 70 Gray-per-second, 80 Gray-per-second, 90 Gray-per-second, or 100 Gray-per-second.

In some examples, ultra-high dose rates of radiation include doses of radiation that exceed one or more of the following doses for a duration of less than 500 ms, for a duration that is between 10 ms and 5 s, or for a duration that is less than 5 s: 100 Gray-per-second, 200 Gray-per-second, 300 Gray-per-second, 400 Gray-per-second, or 500 Gray-per-second.

In some examples, ultra-high dose rates of radiation include doses of radiation that are between 20 Gray-per-second and 100 Gray-per-second for a duration of less than 500 ms. In some examples, ultra-high dose rates of radiation include doses of radiation that are between 20 Gray-per-second and 100 Gray-per-second for a duration that is between 10 ms and 5 s. In some examples, ultra-high dose rates of radiation include doses of radiation that are between 20 Gray-per-second and 100 Gray-per-second for a duration that is less than 5 s. In some examples, ultra-high dose rate rates of radiation include doses of radiation that are between 40 Gray-per-second and 120 Gray-per-second for a time period such as less than 5 s. Other examples of the time period are those provided above.

In some implementations, the particle therapy systems may treat three-dimensional columns of the target using ultra-high dose rate radiation—the FLASH doses of radiation. These systems scale the ultra-high dose rate deliveries to targets using pencil beam scanning. In some examples, pencil beam scanning includes delivering a series of small beams of particle radiation that can each have a unique direction, energy, and charge. By combining doses from these individual beams, a three-dimensional target treatment volume may be treated with radiation. Furthermore, instead of organizing the treatment into layers at constant energies, the systems organize the treatment into columns defined by the direction of a stationary beam. The direction of the beam may be toward the surface of the target.

In some implementations, all or part of a column is treated before the particle beam is directed along another path through the irradiation target. In some implementations, a path through the target is all or part-way through the target. In an example, the particle beam may be directed along a path through a target and not deviate from that path. While directed along that path, the energy of the particle beam is changed. The particle beam does not move as its energy changes and, as a result, the particle beam treats all or a part of an interior portion of the target that extends along a length of the particle beam and along a width of the beam spot. The treatment is thus depth-wise along a longitudinal direction of the beam. For example, a portion of the target treated may extend from a spot of the beam at the surface of the target down through all or part of an interior of the target. The result is that the particle beam treats a three-dimensional columnar portion of the target using an ultra-high dose rate of radiation. In some examples, the particle beam may never again be directed along the same three-dimensional columnar portion more than once.

In some implementations, an irradiation target may be broken into micro-volumes. Although cubical micro-volumes may be used, the micro-volumes may have any appropriate shape, such as three-dimensional orthotopes, regular curved shapes, or amorphous shapes. In this example, each micro-volume is treated through delivery of FLASH radiation by column in the manner described herein. For example, column depths of a micro-volume may be treated with radiation by using energy degrader plates to change the beam energy or by controlling a variable-energy synchrocyclotron to change the beam energy. After an individual micro-volume has been treated, the next micro-volume is treated, and so forth until the entire irradiation target has been treated. Treatment of the micro-volumes may be in any appropriate order or sequence.

The particle therapy system described herein may deliver FLASH radiation by columns in the manner described in U.S. Patent Publication No. 2020/0298025 titled “Delivery Of Radiation By Column And Generating A Treatment Plan Therefor”, the contents of which are incorporated herein by reference, particularly the contents relating to FIGS. 2, 11, 12 to 19, 33 to 43B thereof and the accompanying descriptions.

In some implementations, a particle accelerator other than a synchrocyclotron may be used in the particle therapy system described herein. For example, a cyclotron, a synchrotron, a linear accelerator, or the like may be substituted for the synchrocyclotron in the particle therapy systems described herein.

In some implementations, switching from beam angle to beam angle using the bending magnet may be performed in single-digit or double-digit milliseconds. As a result, radiation delivery time may be reduced. In some examples, by delivering a portion of the beam (one layer or part of one layer) for one angle, range shifters and collimators can reposition, thereby reducing layer switching delays, collimator positioning delays, and treatment angle switching delays.

Operation of the example proton therapy systems described herein, and operation of all or some component thereof, can be controlled, at least in part, using one or more computer program products, e.g., one or more computer programs tangibly embodied in one or more non-transitory machine-readable media, for execution by, or to control the operation of, one or more data processing apparatus, e.g., a programmable processor, a computer, multiple computers, and/or programmable logic components.

All or part of the systems described in this specification and their various modifications may be configured or controlled at least in part by one or more computers, such as control system 34, using one or more computer programs tangibly embodied in one or more information carriers, such as in one or more non-transitory machine-readable storage media. A computer program can be written in any form of programming language, including compiled or interpreted languages, and it can be deployed in any form, including as a stand-alone program or as a module, part, subroutine, or other unit suitable for use in a computing environment. A computer program can be deployed to be executed on one computer or on multiple computers at one site or distributed across multiple sites and interconnected by a network.

Actions associated with configuring or controlling the systems described herein can be performed by one or more programmable processors executing one or more computer programs to control or to perform all or some of the operations described herein. All or part of the systems and processes described herein can be configured or controlled by special purpose logic circuitry, such as, an FPGA (field programmable gate array) and/or an ASIC (application-specific integrated circuit) or embedded microprocessor(s) localized to the instrument hardware.

Processors suitable for the execution of a computer program include, by way of example, both general and special purpose microprocessors, and any one or more processors of any kind of digital computer. Generally, a processor will receive instructions and data from a read-only storage area or a random access storage area or both. Elements of a computer include one or more processors for executing instructions and one or more storage area devices for storing instructions and data. Generally, a computer will also include, or be operatively coupled to receive data from, or transfer data to, or both, one or more machine-readable storage media, such as mass storage devices for storing data, such as magnetic, magneto-optical disks, or optical disks. Non-transitory machine-readable storage media suitable for embodying computer program instructions and data include all forms of non-volatile storage area, including by way of example, semiconductor storage area devices, such as EPROM (erasable programmable read-only memory), EEPROM (electrically erasable programmable read-only memory), and flash storage area devices; magnetic disks, such as internal hard disks or removable disks; magneto-optical disks; and CD-ROM (compact disc read-only memory) and DVD-ROM (digital versatile disc read-only memory).

Elements of different implementations described may be combined to form other implementations not specifically set forth previously. Elements may be left out of the systems described previously without adversely affecting their operation or the operation of the system in general. Furthermore, various separate elements may be combined into one or more individual elements to perform the functions described in this specification.

Other implementations not specifically described in this specification are also within the scope of the following claims.

What is claimed is:

1. A particle therapy system comprising:
a particle accelerator configured to output a particle beam at a predefined maximum energy; and
a toroidal gantry comprising magnets in an interior thereof, the magnets comprising a first magnet proximate to an output of the particle accelerator and second magnets proximate to a treatment position, the first magnet being configured to direct the particle beam to a second magnet, the second magnet being configured to bend the particle at the predefined maximum energy towards the treatment position.

2. The particle therapy system of claim 1, wherein the particle accelerator and the toroidal gantry are within a same treatment space.

3. The particle therapy system of claim 1, wherein the particle accelerator is a fixed-energy particle accelerator; and
wherein the particle therapy system comprises an energy degrader that is movable between each of the second magnets and the treatment position, the energy degrader to change an energy of the particle beam before the particle beam reaches the treatment position.

4. The particle therapy system of claim 1, where the second magnets are spaced apart and are each located in a different circumferential sector of the toroidal gantry.

5. The particle therapy system of claim 4, wherein the toroidal gantry comprises between six and twenty second magnets.

6. The particle therapy system of claim 1, wherein the second magnets are stationary on the toroidal gantry.

7. The particle therapy system of claim 1, wherein the second magnets are configured to bend the particle beam by at least 90°.

8. The particle therapy system of claim 1, further comprising:
a treatment couch that is movable within a hole of the toroidal gantry, the treatment couch for holding a patient at the treatment position.

9. The particle therapy system of claim 1, wherein a distance between the second magnet and the treatment position is two meters (2 m) or less.

10. The particle therapy system of claim 1, wherein a distance between the second magnet and the treatment position is one meter (1 m) or less.

11. The particle therapy system of claim 1, wherein the particle accelerator comprises a synchrocyclotron.

12. The particle therapy system of claim 1, wherein the particle accelerator comprises a synchrocyclotron configured to operate at two energies, one of the two energies being greater than another of the two energies.

13. The particle therapy system of claim 1, wherein the particle accelerator comprises a synchrotron.

14. The particle therapy system of claim 1, further comprising:
one or more imaging devices mounted to the toroidal gantry, the one or more imaging devices being configured for movement around the toroidal gantry.

15. The particle therapy system of claim 14, further comprising:
a nozzle that is configured for movement around the toroidal gantry, the nozzle for outputting the particle beam to the treatment position.

16. The particle therapy system of claim 15, further comprising:
a control system programmed to control movement of the one or more imaging devices and to control movement of the nozzle, the control system being programmed to prevent collision between the nozzle and the one or more imaging devices.

17. The particle therapy system of claim 15, wherein the nozzle is configured to rotate around a first inner track in the toroidal gantry and the one or more imaging device are configured to rotate around a second inner track in the toroidal gantry, the first inner track and the second inner track being at different locations of the toroidal gantry.

18. The particle therapy system of claim 1, where the second magnets are spaced apart and are each located in a different circumferential sector of the toroidal gantry, each of the sectors comprising a nozzle for outputting the particle beam to the treatment position.

19. The particle accelerator of claim 1, wherein the particle accelerator comprise main superconducting coils to generate a magnetic field for accelerating particles to produce the particle beam; and
wherein the particle accelerator comprises active return coils to conduct current in an opposite direction as in the main superconducting coils.

20. The particle accelerator of claim 1, wherein the particle beam is delivered to the patient at FLASH doses.

21. The particle accelerator of claim 20, wherein the particle beam is delivered to the patient at a dose that exceeds twenty (20) Gray-per-second for a duration of less than five (5) seconds.

22. A particle therapy system comprising:
a multi-sectored gantry, each sector being configured to deliver radiation to a patient from a different position on the multi-sectored gantry; and
a particle accelerator connected to the multi-sectored gantry to output the radiation towards the multi-sectored gantry;
wherein the multi-sectored gantry and the particle accelerator are in a same treatment room and not separated by shielding external to the multi-sectored gantry or the particle accelerator.

23. The particle therapy system of claim 22, wherein the multi-sectored gantry and the particle accelerator are in a same treatment space.

24. The particle therapy system of claim 22, wherein each sector comprises a magnet configured to direct the radiation towards the patient.

25. The particle therapy system of claim 24, wherein each magnet is substantially D-shaped.

26. The particle therapy system of claim 24, wherein each magnet is configured to bend the particle beam by at least 90°.

27. The particle therapy system of claim 24, wherein the multi-sectored gantry is toroidal in shape; and
wherein the multi-sectored gantry comprises a second magnet in each sector and a first magnet between the second magnet and the particle accelerator, the first magnet for directing the particle beam to a second magnet in a target sector.

28. The particle therapy system of claim 27, wherein the first magnet is configured to direct the particle beam to different sectors.

29. The particle therapy system of claim 22, wherein the particle accelerator comprises a synchrocyclotron.

30. The particle therapy system of claim 29, wherein the synchrocyclotron is configured to output the particle beam at one of two different energies.

31. The particle therapy system of claim 22, wherein the particle accelerator comprises a synchrotron.

32. A gantry for use in a particle therapy system, the gantry comprising:
a toroidal structure that is connectable to a particle accelerator, the toroidal structure comprising first magnets arranged in sectors around a circumference of the toroidal structure, the first magnets for bending a particle beam originating at the particle accelerator by at least 90° towards a treatment position;
an enclosure connecting the toroidal structure to the particle accelerator, the enclosure comprising second magnets, the second magnets for receiving the particle beam and for directing the particle beam towards the first magnets; and
a rotatable structure within the enclosure configured for mounting at least one of radiation delivery components or imaging components.

33. The gantry of claim 32, the gantry being within a same treatment space as the particle accelerator.

34. The gantry of claim 32, further comprising:
an energy degrader that is movable between each of the first magnets and the treatment position, the energy degrader to change an energy of the particle beam before the particle beam reaches the treatment position, the energy degrader being mounted to the rotatable structure.

35. The gantry of claim 32, where the first magnets are spaced apart and are each located in a different circumferential sector of the toroidal structure.

36. The gantry of claim 32, wherein the toroidal structure comprises between six and twenty first magnets.

37. The gantry of claim 32, wherein the first magnets are stationary on the toroidal structure.

38. The gantry of claim 32, wherein the second magnets are configured to bend the particle beam by at least 90°.

39. The gantry of claim 32, wherein a distance between each of the first magnets and the treatment position is two meters (2 m) or less.

40. The particle therapy system of claim 1, wherein a distance between the second magnet and the treatment position is one meter (1 m) or less.

41. The gantry of claim 32, further comprising:
one or more imaging devices mounted to the rotatable structure, the one or more imaging devices being configured for movement around the toroidal structure.

42. The gantry of claim 32, further comprising:
a nozzle that is configured for movement around the toroidal structure, the nozzle for outputting the particle beam to the treatment position, the nozzle being mounted to the rotatable structure.

43. The gantry of claim 32, further comprising:
one or more imaging devices configured for movement around the toroidal structure; and
a nozzle that is configured for movement around the toroidal structure, the nozzle for outputting the particle beam to the treatment position;
wherein the nozzle and the one or more imaging devices are mounted to the rotatable structure.

44. The gantry of claim 32, where the first magnets are spaced apart and are each located in a different circumferential sector of the toroidal structure, each of the sectors comprising a nozzle for outputting the particle beam to the treatment position.
